(19) United States
(12) Patent Application Publication
(10) Pub. No.: US 2008/0118482 A1
(43) Pub. Date: May 22, 2008

(54) TREATING MOUTH ULCER WITH LIVE BACTERIA

(75) Inventor: Yunlong Cui, Jiaonan (CN)

Correspondence Address: OCCHIUTI ROHLICEK & TSAO, LLP, 10 FAWCETT STREET, CAMBRIDGE, MA, 02138, US

(73) Assignee: Qingdao East Sea Pharmaceuticals, LTD, Jiaonan (CN)

(21) Appl. No.: 11/763,072

(22) Filed: Jun. 14, 2007

(30) Foreign Application Priority Data
Nov. 17, 2006 (CN) 200610138487.5

(51) Int. Cl. A61K 35/74; A61P 1/04

(52) U.S. Cl. 424/93.41

(57) ABSTRACT

This invention relates to using live beneficial bacteria for treating mouth ulcer.

### CROSS-REFERENCE TO RELATED APPLICATIONS

This application claims the benefit of Chinese Patent Application No. 200610138487.5, filed Nov. 17, 2006, the content of which is incorporated herein by reference in its entirety.

### BACKGROUND

Mouth ulcer, one of the most common oral mucous membrane diseases, features an open sore caused by a break in mucous membranes or epithelia on the lips or inside the mouth. It can be extremely painful and often recurs. Due to its multiple causes (e.g., infection, physical trauma, and chemical trauma), no drugs currently available provide satisfactory treatment. Some drugs only relieve pain or suppress ulcer development temporarily, or cause harmful side effects.

Mouth ulcer is found to be associated with a number of medical conditions, such as leukoplakia, oral lichen planus, Crohn's disease, and ulcerative colitis, some of which are digestive disorders.

Certain beneficial bacteria have been found to be effective in treating digestive disorders and diseases known to be accompanied with mouth ulcer, e.g., ulcerative colitis. See US Patent Application 20060127381.

### SUMMARY

The present invention is based on the unexpected discoveries that live beneficial bacteria, such as Bacillus, Clostridium, and Bifidobacterium, are effective in treating mouth ulcer and preventing its recurrence.

In one aspect, treating mouth ulcer is accomplished by first selecting a subject who needs this treatment and then administering to that subject a composition containing an effective amount of a live beneficial bacterium.

The composition described herein can be an orally administrable pharmaceutical composition, or a food product/food supplement. The amount of the live bacterium in this composition ranges from 10⁶-10¹² cfu/g.

The composition can include a live bacterium, such as Bacillus, Clostridium, Bifidobacterium, or a combination of different types of beneficial bacteria. The Bacillus can be Bacillus subtilis, or Bacillus coagulans (e.g. CGMCC No. 1207, deposited at the Chinese General Microbiological Culture Collection Center (CGMCC), Beijing, China.) The Clostridium can be Clostridium butyricum (e.g., CGMCC No. 0313.1.). The Bifidobacterium can be Bifidobacterium adolescentis, Bifidobacterium longum (e.g., CGMCC No. 0313.5), Bifidobacterium bifidum (e.g., CGMCC No. 0313.7), Bifidobacterium breve (e.g., CGMCC No. 0313.6), or Bifidobacterium infantis (e.g., CGMCC No. 0313.2.). The effective amount of these live bacteria is within the range of 10⁶-10¹² cfu per day.

The details of one or more implementations of the invention are set forth in the description below. Other features, objects, and advantages of the invention will be apparent from the description and drawings, and from the claims.

### DETAILED DESCRIPTION

The present invention provides a method of treating mouth ulcer in a subject in need of this treatment with an effective amount of a composition containing a live beneficial bacterium.

The term “mouth ulcer” refers to a break in skin or mucous membrane inside mouth with loss of surface tissue, disintegration and necrosis of epithelial tissue, and often pus. A mouth ulcer typically starts with a tingling or burning sensation at the site of the future mouth ulcer. In a few days, it can progress to form a red spot or bump, followed by an open ulcer. The term “mouth ulcer” used herein includes all stages of a mouth ulcer. It also includes mouth ulcer resulting from various causes, e.g., physical or chemical trauma, bacterial or viral infection, medical conditions or medications. The term “treating mouth ulcer” used herein, unless otherwise indicated, means preventing or inhibiting the formation of mouth ulcer (including red spot/bump or clear open ulcers), relieving the symptoms of mouth ulcer (e.g., pain), reversing, ameliorating, or inhibiting the progress of mouth ulcer, or preventing recurrence of mouth ulcer in a subject.

The term “effective amount” as used herein, refers to an amount or concentration of an agent utilized for a period of time (including acute or chronic administration and periodic or continuous administration) that is effective within the context of its administration for causing an intended effect or physiological outcome. An effective amount of a live beneficial bacterium for use in the present invention include, for example, amounts that are effective for preventing or inhibiting mouth ulcer formation in a subject, or for relieving or ameliorating the symptoms of mouth ulcer, or for preventing mouth ulcer recurrence in a subject.

The term “beneficial bacterium” refers to any bacterium in the intestine and colon that benefits its host in various ways. A beneficial bacterium can form on the top of the intestinal and colonic walls a protective layer, which blocks harmful bacteria and their toxins from damaging or penetrating the walls. Alternatively or in addition, a beneficial bacterium can secret acidic substances (e.g., short-chain fatty acids) resulting in an acidic environment unsuitable for the growth of harmful bacteria. As a result, beneficial bacteria reduce the level of toxins that are generated by harmful bacteria. Moreover, a beneficial bacterium can generate digestive enzymes to decompose food. It can promote digestion by secreting the above-mentioned short-chain fatty acids, which stimulate intestine and colon movement.

The composition for treating mouth ulcer can include one or more types of beneficial bacteria (e.g., B. coagulans, C. butyricum, or a combination thereof). The live beneficial bacteria can be prepared by fermentation carried out under various conditions. One type of bacteria can be cultured individually or co-cultured with another type of bacteria. After the fermentation, the bacteria can be collected by centrifugation and the resultant wet pellets can then be dried by a method that preserves the activity of the bacteria. Suitable drying methods include freeze drying, spray drying, heat drying, or a combination thereof.

The bacteria powder thus obtained can be mixed with a pharmaceutically acceptable carrier to form a pharmaceutical composition. “Acceptable” means that the carrier must be compatible with the active ingredient of the composition (and preferably, capable of stabilizing the active ingredient) and not deleterious to the subject to be treated. Suitable carriers include microcrystalline cellulose, mannitol, glucose, defatted milk powder, polyvinylpyrrolidone, and starch, or a combination thereof. This composition can then be presented in a variety of forms, such as tablet, capsule, powder, or liquid. In this composition, the concentration of the live bacteria is in the range of 10⁶ to 10¹² cfu/g. When the composition contains more than one type of bacteria, the different types of bacteria can be in any ratio, as long as the total amount of bacteria is at least 10⁶ cfu/g.

The live-bacterium-containing composition can be administered to a subject via suitable routes, e.g., oral administration, once or multiple times per day or administered once every several days. A solid formulation for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microglycolate, and alginic acid. Tablet binders that can be used include acacia, methylcellulose, sodium carboxymethylcellulose, polyvinylpyrrolidone (Povidone3), hydroxypropyl methylcellulose, sucrose, starch, and ethylcellulose. Lubricants that can be used include magnesium stearates, stearic acid, silicone fluid, talc, waxes, oils, and colloidal silica.

This solid formulation can be designed such that the composition is released in the intestine. For example, the composition is confined in a solid sub-unit or a capsule compartment that have respectively a matrix or a wall or a closure comprising an enteric polymer which dissolves or disperses at the pH of the small or large intestine to release the drug substance in the intestine. Suitable such polymers have been described above, for example with reference to U.S. Pat. No. 5,705,189.

The bacteria powder also can be part of a food product (e.g., yogurt, milk, or soy milk) or a food supplement (e.g., supply nutrients or herbal products). Such food products can be prepared by methods well known in the food industry.

Effective amount of the live bacterium used in the method described herein can be determined based on factors such as the patient's age, severity and duration of mouth ulcers, and other medical conditions. In general, the effective amount ranges from 10⁶ to 10¹² cfu per day.

The live-bacterium-containing composition described herein can be used in manufacturing medicaments for treating mouth ulcer.

Without further elaboration, it is believed that the above description has adequately enabled the present invention. The following examples are, therefore, to be construed as merely illustrative, and not limitative of the remainder of the disclosure in any way whatsoever. All of the publications cited herein are hereby incorporated by reference in their entirety.

### EXAMPLE 1

### Preparation of Bacteria Powder Containing Live Clostridium butyricum

Clostridium butyricum CGMCC No. 0313.1 stored in a tube was suspended in a 100 ml autoclaved Erlenmeyer flask containing 10 ml physiological saline and suitable amount of glass beads. After 10 minutes, 1 ml bacteria solution was inoculated into a 250 ml Erlenmeyer flask filled with 50 ml amplification media containing tryptone (1%), yeast extract (0.3%), beef exact (1%), glucose (0.5%), soluble starch (0.1%), sodium chloride (0.5%), anhydrous sodium acetate (0.3%), and L-cysteine (0.05%). The bacteria were cultured in a shaker at 37° C. and shaken at the speed of 190 rpm for 24 hours. Then the bacteria solution was transferred to a 2500 ml baffled Erlenmeyer flask containing 450 ml amplification media, cultured at 37° C. with shaking for another 24 hours. The bacteria solution was examined under microscope for contamination. If no contamination had occurred, the bacteria solution was transferred into a seeding tank containing 4.5 L amplification media and further cultured under aerobic conditions (air inflation amount 3:1) for yet another 24 hours. The resulting bacteria solution, if not contaminated, was transferred to a fermentation tank filled with fermentation media and cultured under aerobic conditions for 24 hours. When the sporulation rate reached 80% (determined by microscopic examination), the fermentation was terminated. The bacteria were collected by centrifuging at 12,000 rpm. Wet bacteria pellets were collected and weighed. The same amount (by weight) of defatted milk powder was mixed with the bacteria, dried, pulverized and kept at room temperature ready for use.

### EXAMPLE 2

### Preparation of Live Clostridium butyricum Capsule

The following is a formulation of live Clostridiumm butyricum capsule:

| Components | % (Weight) |
|---|---|
| Live bacterial powder of Clostridium butyricum | 20.00 parts |
| Microcrystalline cellulose | 40.00 parts |
| Glucose | 60.00 parts |

The above-described three components were mixed completely and made into capsules in unit dosage according to conventional encapsulating technology.

### EXAMPLE 3

### Using Live Bacteria of Bacillus, Clostridium and Bifidobacterium for Treating Mouth Ulcer and Preventing Its Recurrence

Fifteen mouth ulcer patients (10 male, 5 female, average age 38) were selected for this study. All of these patients had a history of suffering from mouth ulcer for averagely 2-10 years. In each patient, mouth ulcer had recurred at least six times per year.

These patients were divided into three groups. Each group was orally administered with Clostridium butyricum CGMCC 0313.1 capsules (420 mg per capsule, three capsules each time, twice per day), Bacillus coagulans CGMCC 1207 tablets (350 mg per tablet, three tablets each time, three times per day), or Bifidobacterium infantis CGMCC 0313.2 tablets (350 mg per tablet, three tablets each time, three times per day), for two weeks. In each capsule or tablet, the amount of the live bacterium is at least 1.0×10⁶ cfu/g.

The efficacy of treating mouth ulcer was determined by the following standards:
Highly Effective: Within three days after the treatment, symptoms of ulcer disappear or areas of ulcer shrink; pain disappears.
Effective: Within five days after the treatment, symptoms of ulcer disappear or areas of ulcer shrink; pain disappears.
Ineffective: Symptoms disappear or areas of ulcer shrink seven days after the treatment.

The efficacy of preventing mouth ulcer recurrence was determined by the following standards:
Highly Effective: No mouth ulcer recurrence occurs within three months after live bacteria treatment.
Effective: No mouth ulcer recurrence occurs within two months after live bacteria treatment.
Ineffective: Mouth ulcer recurrs within two months after live bacteria treatment.

All patients were free from mouth ulcer and pain five days after taking the live bacteria. The overall cure rates of all three groups were 100%. After the treatment, these patients were followed up for possible mouth ulcer recurrence for three months. None had recurred mouth ulcer within this period. No patients showed any side effects after treated.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of treating mouth ulcer, the method comprising:
identifying a subject in need thereof, and
administering to the subject a composition which contains an effective amount of a live beneficial bacterium.

2. The method of claim 1, wherein the composition contains Bacillus, Clostridium, Bifidobacterium, or a combination thereof.

3. The method of claim 2, wherein the Bacillus is Bacillus coagulans.

4. The method of claim 3, wherein the Bacillus is Bacillus coagulans CGMCC No. 1207

5. The method of claim 1, wherein the Clostridium is Clostridium butyricum.

6. The method of claim 5, wherein the Clostridium is Clostridium butyricum No. 0313.1.

7. The method of claim 1, wherein the Bifidobacterium is Bifidobacterium infantis, Bifidobacterium longum, Bifidobacterium breve, or Bifidobacterium bifidum.

8. The method of claim 7, wherein the Bifidobacterium is Bifidobacterium infantis CGMCC No. 0313.2, Bifidobacterium longum CGMCC NO. 0313.5, Bifidobacterium breve CGMCC No. 0313.06, or Bifidobacterium bifidum CGMCC No. 0313.7.

9. The method of claim 1, wherein the effective amount of the live beneficial bacterium is within the range of 10⁶ to 10¹² cfu per day.

10. The method of claim 1, wherein the composition is administered orally.

11. The method of claim 1, wherein the composition is a pharmaceutical composition.

12. The method of claim 11, wherein the pharmaceutical composition is formulated in a form of tablet, capsule, powder, or liquid.

13. The method of claim 11, wherein the pharmaceutical composition contains 1×10⁶ CFU/g to 1×10¹² CFU/g live beneficial bacteria.

14. The method of claim 1, wherein the composition is a food product or a food supplement.
